(19) United States
(12) Patent Application Publication
(10) Pub. No.: US 2008/0249572 A1
(43) Pub. Date: Oct. 9, 2008

(54) Bone Plate

(76) Inventor: Vineet Dev Tandon, Cheshire (GB)

Correspondence Address: DENNISON, SCHULTZ & MACDONALD, 1727 KING STREET, SUITE 105, ALEXANDRIA, VA, 22314, US

(21) Appl. No.: 11/579,185

(22) PCT Filed: May 19, 2005
(86) PCT No.: PCT/GB2005/001933

§ 371 Date: Nov. 30, 2006

(30) Foreign Application Priority Data
Jun. 2, 2004 (GB) 0412222.2

(51) Int. Cl. A61B 17/80

(52) U.S. Cl. 606/280

(57) ABSTRACT

A bone plate (15) is provided with a keyhole shaped aperture (10) having a first wide portion (20) leading into a second narrow portion (22) by way of a discontinuous narrowing (24). The narrow portion (22) has a concave recess (26) in the upper surface (11) of the plate and a bevel (28) in the bone contacting surface (13) at its end remote from the wide portion. In use, a screw (30) can be inserted obliquely across a fracture (40) and does not need to be countersunk in the bone, nor removed and replaced to allow fitting of the plate (15). The wide portion (20) of the hole (10) passes over the head (32) of the screw (30), the plate is then slid (arrow a) so that the head (34) lies in the narrow portion (22). Once the screw is fully tightened its head sits in the concave recess (26) and because of the narrowing (24), it prevents the plate sliding back. The screw shank (34) adjacent the head is accommodated in the bevel (28). Further bone screws can then be secured via other apertures (12, 14, 16, 18) in the plate (15).

The present invention relates to a bone plate for use in surgery for securing across a fracture in a bone.

A bone plate comprises a plate of bio-acceptable material, such as titanium or a stainless steel alloy, with holes in the plate. The holes are used to attach the plate to a bone by means of bone securing screws (‘screws’). Screws are placed through the plate into the bone either side of a fracture between broken ends of the bone. The broken ends are therefore stopped from moving relative to one another during the healing process. After healing the bone plate can be removed.

The above technique is rarely satisfactory for oblique fractures, i.e. fractures that are not perpendicular to the main axis of the bone, as a sliding wedge action can occur whereby, on securing the bone plate with the screws, either the bone surface is held level and the fracture left open or the fracture held closed and the bone surface left offset.

Direct fixation of a screw across a fracture to exert a compressive force between the respective fragments assists in quicker bone healing. This is often referred to as inter-fragmentary fixation and involves the holding of bone fragments in a satisfactory alignment using a clamp followed by drilling across the fracture to create a hole for a screw.

In a first method the hole created in a near fragment is made wider so that the screw thread of the shank of the screw only holds the far fragment. Hence, when the screw is tightened the fracture is compressed between the screw head on the near fragment and the screw shank holding the far fragment. A second method uses screws that are threaded in the distal part only. As these have no purchase in the proximal fractured fragment, as the screw is tightened the fracture gap is diminished by compression achieved between the screw head on the proximal fragment and screw threads holding the distal fragment.

Whilst advantageous, inter-fragmentary screw fixation is problematic when used with a bone plate to further support the bone. In particular the screw head of the inter-fragmentary screw obstructs the placing of the plate on the bone. Two known, but non-optimal techniques are currently used to limit this problem.

In a first known method, the screw head is countersunk into the bone to allow a bone plate to be placed over the inter-fragmentary screw. This adds another step to the operation, weakens the countersunk bone fragment and makes subsequent removal of this screw difficult, as bone tends to grow over the screw head.

The other known method is to remove the originally installed inter-fragmentary screw and then clamp to reduce (i.e. close) the fracture with a plate over it and hold the bones as well as the plate together. The original screw or a replacement can then be placed through a hole in the plate and back down the pre-existing drill hole across the fracture. This method is technically demanding as it requires holding three independently mobile pieces, the two bone fragments and the plate, all with one clamp. Moreover, it is often difficult to be sure that the fracture is reduced as the bone plate usually overlies and hides the relevant part of the bone underneath it.

It is an aim of the invention to provide a bone plate whereby inter-fragmentary screw fixation for securing a broken bone can be more readily combined with the use of a bone plate.

A first aspect of the present invention provides a bone plate comprising an upper surface, an opposing bone contacting surface, and at least one keyhole shaped aperture extending through the upper and bone contacting surfaces for receiving a bone screw, the at least one keyhole shaped aperture being elongated to define a major axis and having first and second portions at respective ends of the major axis, the first portion having a minor axis, perpendicular to the major axis, which is greater than the maximum width, also perpendicular to the major axis, of the second portion, and the second portion extending from the first portion via a discontinuous narrowing in the width of the aperture, characterised in that no portion of the keyhole shaped aperture has an internal thread and in that the second portion includes a concave recess formed in the upper surface adjacent the first portion and a bevel formed in the bone contacting surface remote from the first portion so as to be capable of receiving a head and adjoining shank portion of a screw which extends at an acute angle to the plate with the head seated in the concave recess and the adjoining shank portion accommodated in the bevel.

Usually only one such keyhole shaped aperture is provided in a bone plate, but there could be more than one in some embodiments.

Additional apertures which are substantially circular in shape or oblong, i.e. slightly longer than wide, as are provided in conventional bone plates, may also be present. Oblong or elongate apertures are preferable as the increased length compared with the width of an aperture enables a screw to be accommodated even if it is inserted through the aperture at a slight angle. The upper surface of the bone plate surrounding each such aperture may be bevelled or formed as a concave recess. This allows heads of additional screws which are applied through these additional apertures to be at least partially countersunk into the plate.

The bone plate of the invention allows a surgical procedure to be performed in which an inter-fragmentary screw is used across an oblique fracture. The screw is inserted at an angle to the bone structure. It is not fully tightened, i.e. not inserted to maximum extent, and the screw head remains proud of the bone surface. The bone plate is then placed over the screw head with the wider first portion of the keyhole aperture passing over the screw head. The bone plate is then slid along substantially in abutment with the bone so as to locate the screw shank portion which is proud of the bone in the bevelled end of the second narrower portion of the aperture. The screw is then secured, fully, by inserting to its maximum extent by one or a few final turns. This brings the screw head fully into the concave recess region of the second portion so that it is partially countersunk into the plate. The constriction between the first and second portions of the keyhole aperture retains the screw in this position, preventing any further sliding of the plate. Thus the screw compresses the bone across the fracture and also secures the plate to the bone. Further screws are then placed through the customary circular or elongate securing apertures to fully secure the plate to the bone.

If more than one keyhole aperture is present the procedure remains the same. The screws to be accommodated in the respective keyhole apertures are all applied at the same stage of the procedure. However, it is preferred that only one keyhole aperture is present as otherwise it is troublesome to reliably achieve the requisite spacing between the screws.

Use of a bone plate of the invention enables the disadvantages of known techniques to be avoided and reduces the time taken for surgery and its complexity. As compared with the first technique mentioned above use of the bone plate of the invention avoids the step of counter sinking the screw head in the bone and thus avoids weakening the bone. It also makes subsequent screw removal, after bone healing, much easier than with that earlier technique. The bone plate of the invention is also helpful in parts of bones that have a thin cortex such as cancellous bone or bone in which osteoporosis has occurred. Use of the bone plate of the invention as compared to the second technique mentioned above avoids removal of a screw that has already been placed. It is therefore technically less demanding as a clamp is only required to hold the two fragments of bone during the initial inter-fragmentary screw application.

In some embodiments of the bone plate of the invention the first and second portions of the keyhole shaped aperture may each describe part of a perimeter of a respective circle so that the outline of the keyhole aperture is similar to that of a figure of 8 or a compressed or squashed figure of 8.

In other embodiments the or each keyhole shaped aperture may comprise a wider first portion with edges which describe a part of the perimeter of a circle and a narrower second portion which is elongated and has a curving end remote from the wide aperture portion. The elongated narrower second portion may have substantially parallel side walls and the end remote from the first portion describing a semicircle. However, it may have converging side walls. In embodiments having an elongated second portion, the concave recess in the upper surface, adjacent the first portion, is still present, but the bevel in the under surface at the end remote from the first portion is not essential so long as the second portion is long enough to accommodate an obliquely extending screw shank with the screw head seated in the concavity. This variant is, accordingly, a second aspect of the invention. It is used in the same way as already described, and has the same advantages and possibilities for additional apertures as embodiments according to the first aspect.

In yet other embodiments, in accordance with third and fourth aspects of the invention, the or each keyhole shaped aperture has a central, wider first portion and respective narrower second portions extending therefrom at opposing ends of the major axis of the aperture via respective discontinuous constrictions in the width of the aperture. Once again, each second portion has a concave recess formed in the upper surface of the plate adjacent the first portion. However, one version includes a bevel formed in the bone contacting surface at the end of each second portion remote from the first portion, while the other version simply has each second portion sufficiently elongated as to be capable of receiving a head and adjoining shank portion of a screw which extends at an acute angle to the plate. These two forms could, of course, be combined, so one second portion has a bevel while the other is merely elongated.

Again these further versions having a central first portion would be used in just the same way as already described, with the same advantages and possibilities for additional apertures as the first version mentioned above, except that they could obviously cater for a screw inserted into a bone at an inclination in either direction.

A bone plate of the invention may be substantially rectangular, it may be elongate and it may be shaped to fit a particular bone.

The plate may be wider around the wider first portion of the keyhole aperture to ensure adequate strength at that location where the remaining plate material may be narrower. However, it may be preferable to make the entire plate slightly wider than is conventional and maintain its rectangular shape.

Bone plates of the invention may be flat plates or may have a concave under side and/or a convex upper side, i.e. the plate may be planar or it may be of a curved cross-section.

In embodiments where there is more than just a single keyhole aperture, the apertures in the plate for receiving screws, i.e. the keyhole aperture(s) and any further circular or elongate apertures at either side of the keyhole aperture(s) may all lie on a straight line along the plate and may be evenly spaced along the bone plate.

In other embodiments the various screw receiving apertures in the plate may be positioned along the length and width of the plate in any pattern best suited to the bone where the plate is to be used and the nature of the fracture.

Apertures other than the aforesaid screw receiving apertures are unlikely to be present as they would adversely affect the strength of the plate.

All the circular or elongate securing apertures may be of the same diameter or width. The wider first portion of the keyhole shaped aperture is preferably of greater diameter than any of the circular or elongate securing apertures. The narrower second portion (or portions) of the keyhole aperture may be of a smaller, or may be of an equal, maximum width to the circular or elongate securing apertures. The latter can be advantageous, as the use of only one size of screw for all securing apertures is then possible, thus reducing the possibility of errors in an operation using the plate.

The or each keyhole aperture preferably has smooth sides and, in particular, has no screw threaded portion. The use of screw threads in a bone plate is disadvantageous. It makes subsequent removal of a screw difficult, as bone tends to grow into the thread.

The invention may be supplied as a kit comprising a bone plate and a number of bone screws.

The invention will be described further, by way of example, with reference to the accompanying drawings, in which;

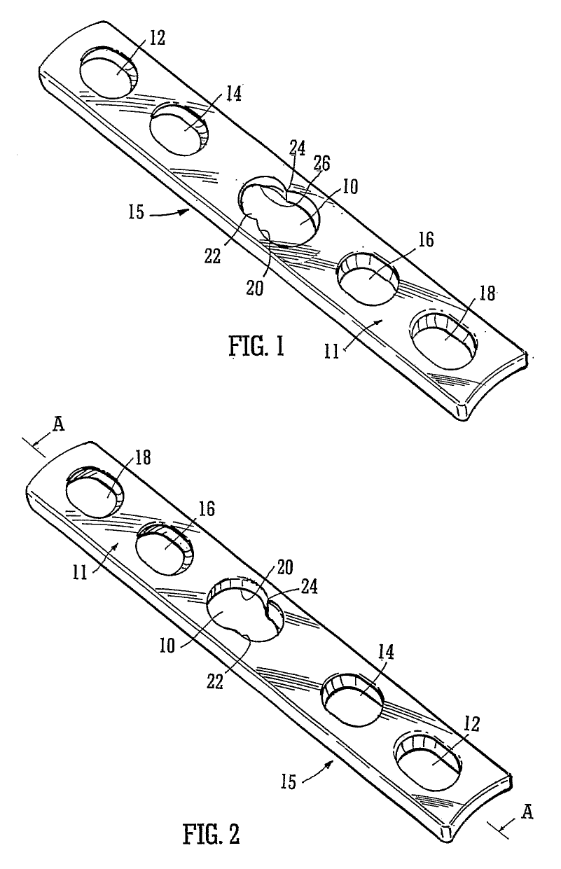

FIG. 1 is a perspective view from above and one side of a first practical embodiment of a bone plate according to the invention;

FIG. 2 is a similar view showing the same bone plate but from the other side as compared to FIG. 1;

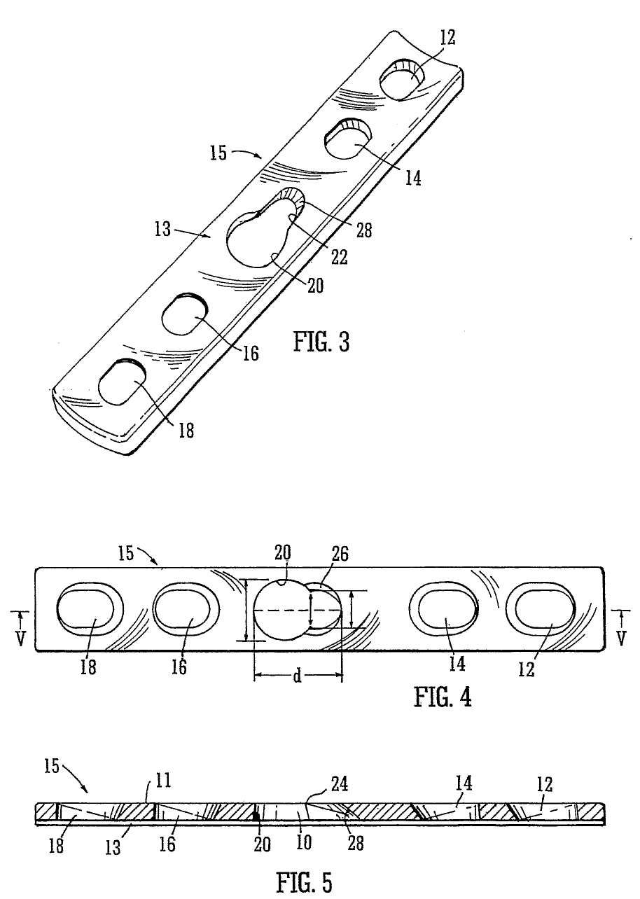

FIG. 3 is a perspective view from below of the same bone plate as in FIGS. 1 and 2;

FIG. 4 is a plan view of the same bone plate;

FIG. 5 is a cross-section of the same bone plate along the line V-V in FIG. 4;

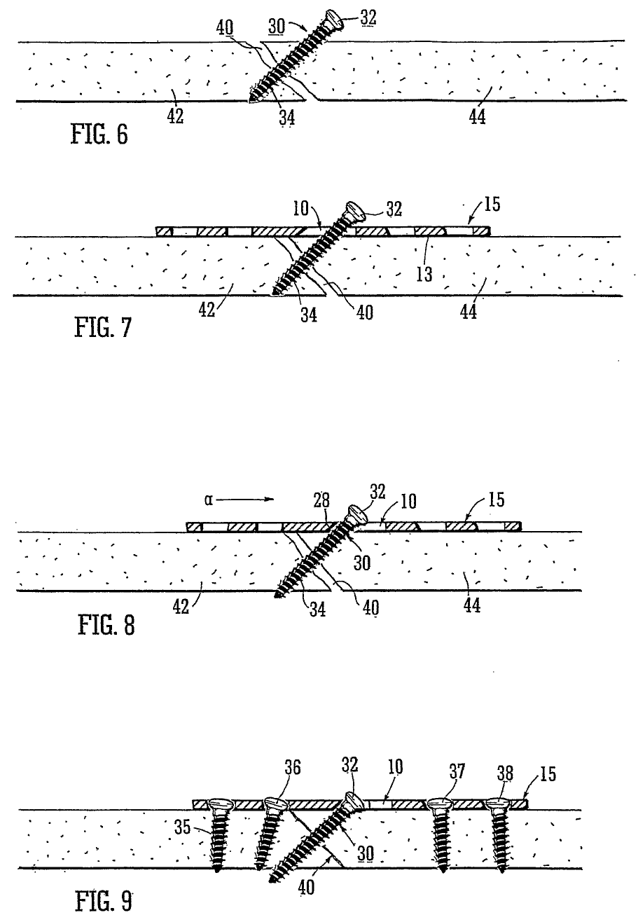

FIGS. 6 to 9 illustrate sequential steps in surgical use of the bone plate shown in the preceding Figures;

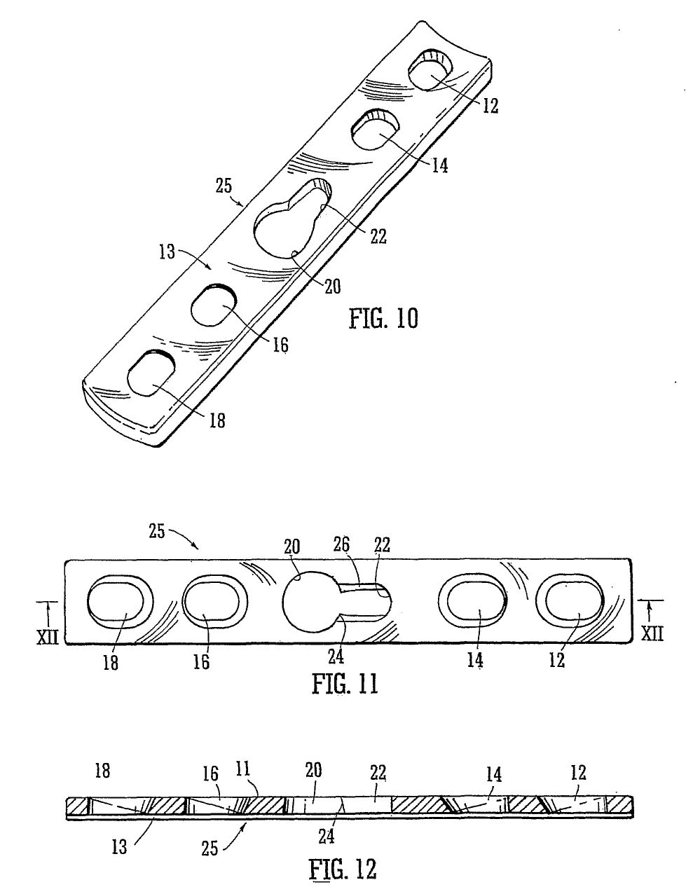

FIG. 10 is a perspective view from below of a second practical embodiment of the bone plate of the invention;

FIG. 11 is a plan view of the second embodiment;

FIG. 12 is a cross-section of the second embodiment along line XII-XII in FIG. 11;

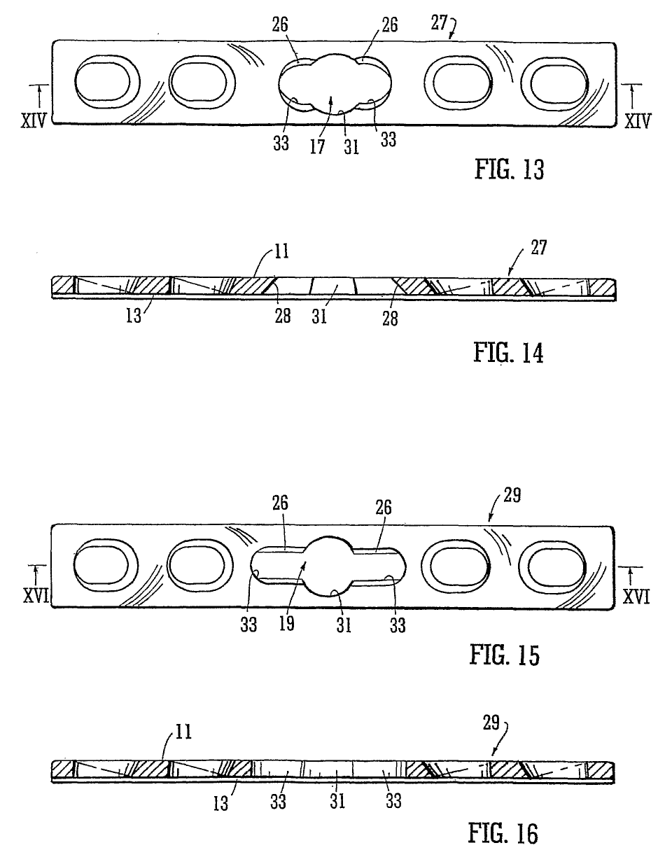

FIG. 13 is a plan view of a third embodiment of the bone plate of the invention;

FIG. 14 is a cross-section of the third embodiment along line XIV-XIV in FIG. 13;

FIG. 15 is a plan view of a fourth embodiment of the bone plate of the invention; and

FIG. 16 is a cross-section of the fourth embodiment along line XVI-XVI in FIG. 15.

Referring to FIGS. 1 to 5 of the drawings, a first practical embodiment 15 of a bone plate of the invention is made of biocompatible stainless steel alloy and has a keyhole shaped aperture 10 and four elongate but rounded securing apertures 12, 14, 16, 18 as are known in the prior art. The plate 15 has an upper surface 11, as seen in FIGS. 1, 2 and 4, and a bone contacting surface 13, which is its underside, as seen in FIG. 3. The bone plate 15 is an elongate rectangle in plan view (FIG. 4) and the apertures 10-18, which all extend between the upper and bone contacting surfaces 11, 13 are arranged in a row along a longitudinal axis A of the plate 15. The keyhole shaped aperture 10 lies in the middle, with two of the known style of securing apertures (12, 14; 16, 18) at each side, towards each end of the plate 15.

The keyhole shaped aperture 10 has a first wide aperture portion 20 leading into a second narrower portion 22 by way of distinct, discontinuous narrowing or constriction 24 in the width of the aperture 10. The wide aperture portion 20 has a substantially part-circular periphery and its bore extends perpendicular to the upper and bone contacting faces 11, 13 of the bone plate 15. In other words, the sides of the wide portion 20 are straight, not bevelled at all, as there is no reason for bevelling. The narrow aperture portion 22, in this case, is somewhat elongated. Adjacent the first portion 20, a concave recess 26, which is most apparent in FIGS. 1 and 4, is formed in the upper surface 11 around the second, narrower aperture portion 22 and this gives it the appearance of having a part-circular shape in its upper region.

Additionally, a significant bevel 28 is formed in the bone contacting surface 13 at the end of the narrow portion 22 remote from the wide portion 20, as is apparent in FIGS. 3 and 5.

For purposes of later explanation and definition, the keyhole shaped aperture 10 is shown in FIG. 4 as having a major axis of length d, a minor axis, perpendicular thereto, of length e, which is the diameter of the wide portion 20, a maximum width f of the narrow portion 22, which is also perpendicular to the major axis, and a width g at the constriction 24, which is perpendicular to the major axis.

The maximum width f of the narrow portion does not include the concave recess 26 in the upper surface 11. The length d does not include the bevel 28 in the bone contacting surface 13.

In all cases e>f≧g. Expressed in words, the diameter (or maximum width when not part-circular) of the wide portion 20 is always greater than the diameter (or maximum width when not part-circular) of the narrow portion 22. The constriction is usually narrower than the latter, but may be equal thereto when the narrow portion itself is elongate, not part-circular.

The plate is curved transversely, the curve being visible in FIGS. 1 to 3.

The narrow portion 22 of the keyhole shaped aperture has a maximum width f substantially the same as that of the elongate securing apertures 12, 14, 16, 18 while the wide aperture portion 20 has a diameter greater than that of the apertures 12, 14, 16, 18.

In use, as illustrated in FIGS. 6 to 9, an inter-fragmentary bone screw 30 having a head 32 with a hexagonal recessed hole (not shown) for receiving a screwdriver, and a shank 34, is inserted across a fracture 40 between two fragments 42, 44 of a broken bone. The screw 30 is located in a pre-drilled hole across the fracture 40 at an acute angle to the surface of the bone. After securing the screw 30 across the fracture 40, but not fully tightening it, the bone plate 15 of FIGS. 1 to 5 is placed over the screw head 32 with its lower face 13 lying on the bone surface. Specifically, the wide portion 20 of the keyhole aperture 10 passes over the screw head 32 with reasonable clearance to achieve the position shown in FIG. 7. The bone plate 15 is then slid longitudinally in the direction of arrow a in FIG. 8 such that the narrow portion 22 of the keyhole aperture 10 surrounds the shank 34 of the screw adjacent the head 32 and the inclination of the shank 34 is accommodated in the bevel region 28, as is apparent in FIG. 8. The screw 30 is then tightened to bring the head 32 into a partially countersunk position within the concave recess 26 of the narrow portion 22 of the keyhole aperture 10. Upon tightening of the screw the fracture 40 is compressed (closed), the tip of the screw shank 34 may project from the other side of the bone, as shown in FIG. 9, and the plate 15 is held in place. Also, because the head 32 is now seated at an angle in the narrow portion 22 of the aperture, beyond the constriction 24, the plate 15 is prevented by the constriction 24 from sliding back relative to the bone. Four further screws 35, 36, 37, 38 are then secured, respectively, through the elongate securing apertures 12, 14, 16, 18 to complete the attachment of the bone plate 15 to the bone 42, 44.

After the fracture 40 has healed the bone plate 15 is readily removable by unscrewing the screws 30, 35, 36, 37, 38.

The bone screws are available in a range of sizes and the dimensions of the keyhole shaped aperture will be adapted to suit particular sizes of screw.

A commonly used bone screw has a head diameter of 6 mm and a shank diameter of 3.5 mm. Suitable dimensions for the keyhole aperture may then be

e=6.5 mm
f=4.5 mm
g=4 mm

The maximum width of the wide portion is then 31% greater than the maximum width of the narrow portion, ((e−f)/e)×100%) and 38.5% greater than the width at the constriction.

A particularly large bone screw may have a head diameter of 8 mm and a shank diameter of 6.5 mm. Suitable dimensions for the keyhole aperture may then be

e=8.5
f=7.5
g=7.0

The maximum width of the wide portion is then 11.75% greater than the maximum width of the narrow portion and 17.5% greater than the width of the constriction.

A particularly small bone screw may have a head diameter of 3 mm and a shank diameter of 1.5 mm. Suitable dimensions for the keyhole aperture may then be

e=3.5
f=2.5
g=1.6

The maximum width of the wide portion is then 28% greater then the maximum width of the narrow portion and 54% greater than the width of the constriction.

Generally speaking the wide portion will be at least 10%, in some cases over 20% and up to 50% or more wider at its maximum than the narrow portion. The constriction will always be narrower when the narrower portion is part-spherical and may be equal or narrower when the narrow portion is of an elongated form.

FIGS. 10 to 12 show an alternate embodiment of bone plate 25 which differs from the one already described by the absence of the bevel 28 in the under surface 13 at the end of the second portion 22 and by otherwise having a somewhat more elongated second portion 22, with the concavity 26 in it's upper surface 11 also extended, but not around the end. The same reference numerals have been used to designate parts corresponding to those of the first embodiment, and further detailed description is not necessary. This plate 25 is used in exactly the same manner as the first embodiment. The elongated end of the narrow portion 22 accommodates the part of the shank 34 adjoining the head 32 of the screw when this plate is used and the stages in the procedure as shown in FIGS. 8 and 9 are reached.

FIGS. 13 to 16 show two further embodiments of bone plate, designated 27 and 29, respectively. These have differently shaped keyhole apertures, 17 and 19 respectively, which have a longer major axis than the preceding embodiments and have a central first, wider portion 31 and two second narrower portions 33 at opposing sides thereof. In the embodiment shown in FIGS. 13 and 14, the second portions 33 are each of the same form as in FIGS. 1 to 5, including a concavity 26 in the upper surface of the plate and a bevel 28 in the bone contacting surface at the end remote from the wide portion. In the embodiment shown in FIGS. 15 and 16, the second portions 33 are each of the same form as in FIGS. 10 to 12, being elongated and lacking the aforesaid bevel in the under surface. These are both used in corresponding manner to the preceding embodiments, but cater for the screw 30, as inserted into the bone 42,44, being inclined in either direction.

The foregoing is illustrative and not limitative of the scope of the invention and many variations in detail are possible. For example, in other embodiments the shape of the plate may vary, depending on the bone and the site on the bone where it is to be used, the position of the keyhole shaped aperture may vary with the shape of the plate, the exact configuration of the keyhole shaped aperture may vary, and the number, position and configuration of other securing apertures may vary.

1-15. (canceled)

16. A bone plate comprising
an upper surface;
an opposing bone contacting surface; and
at least one keyhole shaped aperture extending through the upper and bone contacting surfaces for receiving a bone screw;
the at least one keyhole shaped aperture being elongated to define a major axis and having first and second portions at respective ends of the major axis, the first portion having a minor axis, perpendicular to the major axis, which is greater than the maximum width, also perpendicular to the major axis, of the second portion, and the second portion extending from the first portion via a discontinuous narrowing in the width of the aperture;
wherein no portion of the keyhole shaped aperture has an internal thread; and
wherein the second portion includes a concave recess formed in the upper surface adjacent the first portion and a bevel formed in the bone contacting surface remote from the first portion so as to be capable of receiving a head and adjoining shank portion of a screw which extends at an acute angle to the plate with the head seated in the concave recess and the adjoining shank portion accommodated in the bevel.

17. A bone plate according to claim 16 wherein the minor axis of the first portion is at least 10% greater than the maximum width of the second portion.

18. A bone plate according to claim 16 wherein the minor axis of the first portion is at least 15% greater than the discontinuous narrowing in the width of the aperture.

19. A bone plate according to claim 16 wherein the minor axis of the first portion is at least 20% greater than the maximum width of the second portion.

20. A bone plate according to claim 16 wherein the minor axis of the first portion is at least 25% greater than the discontinuous narrowing in the width of the aperture.

21. A bone plate according to claim 16 wherein the first portion has a substantially part-circular outer periphery.

22. A bone plate according to claim 16 wherein the first portion has an outer periphery extending substantially perpendicular between the upper and bone contacting surfaces.

23. A bone plate according to claim 16 wherein the second portion has a substantially part-circular outer periphery.

24. A bone plate according to claim 16 wherein further apertures are provided, extending through the upper and bone contacting surfaces for receiving respective bone screws, these further apertures being substantially circular or elongate in shape and having a diameter or width which is less than the minor axis of the first portion of the keyhole shaped aperture.

25. A bone plate according to claim 16 wherein further apertures are provided, extending through the upper and bone contacting surfaces for receiving respective bone screws, these further apertures being substantially circular or elongate in shape and having a diameter or width which is substantially equal to the maximum width of the second portion of the keyhole shaped aperture.

26. A bone plate comprising
an upper surface;
an opposing bone contacting surface; and
at least one keyhole shaped aperture extending through the upper and bone contacting surfaces for receiving a bone screw, the at least one keyhole shaped aperture being elongated to define a major axis and having a first central portion and respective second portions at respective ends of the major axis, the first portion having a minor axis, perpendicular to the major axis, which is greater than the maximum width, also perpendicular to the major axis, of each of the second portions, and each of the second portions extending from the first portion via a respective discontinuous narrowing in the width of the aperture;
wherein no portion of the keyhole shaped aperture has an internal thread and wherein at least one of the second portions includes a concave recess formed in the upper surface adjacent the first portion and a bevel formed in the bone contacting surface remote from the first portion so as to be capable of receiving a head and adjoining shank portion of a screw which extends at an acute angle to the plate with the head seated in the concave recess and the adjoining shank portion accommodated in the bevel.

27. A bone plate according to claim 26 wherein the minor axis of the first portion is at least 10% greater than the maximum width of each second portion.

28. A bone plate according to claim 26 wherein the minor axis of the first portion is at least 15% greater than each discontinuous narrowing in the width of the aperture.

29. A bone plate according to claim 26 wherein the minor axis of the first portion is at least 20% greater than the maximum width of each second portion.

30. A bone plate according to claim 26 wherein the minor axis of the first portion is at least 25% greater than each discontinuous narrowing in the width of the aperture.

31. A bone plate according to claim 26 wherein the first portion has a substantially part-circular outer periphery.

32. A bone plate according claim 26 wherein the first portion has an outer periphery extending substantially perpendicular between the upper and bone contacting surfaces.

33. A bone plate according to claim 26 wherein each second portion has a substantially part-circular outer periphery.

34. A bone plate according to claim 26 wherein further apertures are provided, extending through the upper and bone contacting surfaces for receiving respective bone screws, these further apertures being substantially circular or elongate in shape and having a diameter or width which is less than the minor axis of the first portion of the keyhole shaped aperture.

35. A bone plate according to claim 26 wherein further apertures are provided, extending through the upper and bone contacting surfaces for receiving respective bone screws, these further apertures being substantially circular or elongate in shape and having a diameter or width which is substantially equal to the maximum width of each second portion of the keyhole shaped aperture.

36. A bone plate comprising
an upper surface;
an opposing bone contacting surface; and
at least one keyhole shaped aperture extending through the upper and bone contacting surfaces for receiving a bone screw, the at least one keyhole shaped aperture being elongated to define a major axis and having first and second portions at respective ends of the major axis, the first portion having a minor axis, perpendicular to the major axis, which is greater than the maximum width, also perpendicular to the major axis, of the second portion, and the second portion extending from the first portion via a discontinuous narrowing in the width of the aperture;
wherein no portion of the keyhole shaped aperture has an internal thread; and
wherein the second portion includes a concave recess formed in the upper surface adjacent the first portion and is sufficiently elongated as to be capable of receiving a head and adjoining shank portion of a screw which extends at an acute angle to the plate with the head seated in the concave recess and the adjoining shank portion accommodated in the elongated end region of the second portion.

37. A bone plate comprising
an upper surface;
an opposing bone contacting surface; and
at least one keyhole shaped aperture extending through the upper and bone contacting surfaces for receiving a bone screw, the at least one keyhole shaped aperture being elongated to define a major axis and having a first central portion and respective second portions at respective ends of the major axis, the first portion having a minor axis, perpendicular to the major axis, which is greater than the maximum width, also perpendicular to the major axis, of each of the second portions, and each of the second portions extending from the first portion via a discontinuous narrowing in the width of the aperture;
wherein no portion of the keyhole shaped aperture has an internal thread; and
wherein at least one of the second portions includes a concave recess formed in the upper surface adjacent the first portion and is sufficiently elongated as to be capable of receiving a head and adjoining shank portion of a screw which extends at an acute angle to the plate with the head seated in the concave recess and the adjoining shank portion accommodated in the elongated end region of the second portion.
